(19) United States
(12) Patent Application Publication
(10) Pub. No.: US 2024/0050616 A1
(43) Pub. Date: Feb. 15, 2024

(54) Device and method for dispensing and/or diffusing volatile substances, especially for dispensing and/or diffusing fragrances and/or active substances

(71) Applicant: CTR, LDA, Lotes 23/24 (PT)

(72) Inventors: David Dycher, Port Erin (IM); Pedro Queiroz Vieira, Belas (PT); Harold Augier, Zhongshan (CN)

(21) Appl. No.: 18/259,149

(22) PCT Filed: Sep. 11, 2020
(86) PCT No.: PCT/EP2020/081436

§ 371 Date: Jun. 23, 2023

(51) Int. Cl. A61L 9/03

(52) U.S. Cl. CPC A61L 9/037; A61L 9/032; A61L 2209/135

(57) ABSTRACT

The invention relates to a device for dispensing and/or diffusing volatile substances, comprising a housing (32), a storage tank (33) and an electrical heating device (30). The device further comprises a capillary element (34) which is coupled to the heating element in a heat-transferring manner, the capillary element (34) having a substance-receiving area (35) which is in contact with a substance received in the storage tank (33) and which substance is conveyable to a substance-discharging area (36) of the capillary element (34) at which the substance is dispensed. Furthermore, an air inlet (50) is provided, by means of which ambient air (53) enters from outside the housing (32) and forms a pure air flow (40) in the housing interior. An air duct (54) is provided which, starting from the air inlet (50), extends as far as the substance discharging area (36), at which a substance air flow (51) enriched with substance is generated, and which, starting from the substance discharging region (36), is led to an air outlet (38) via which the substance air flow (51) is discharged to the environment. In addition, a control device (39) is provided, by means of which the electrical heating device (30) can be controlled, preferably at intervals.

The invention relates to a device for dispensing and/or diffusing volatile substances, in particular for dispensing and/or diffusing fragrances and/or active substances, according to the preamble of claim 1, and a method for operating a device for dispensing volatile substances, in particular for dispensing fragrances and/or active substances, according to the preamble of claim 23.

Devices for dispensing volatile substances are well known and usually comprise a container in which a substance to be dispensed is contained. For example, the container contains a capillary wick which protrudes above the container with a free end of the wick and which is in contact with the substance to be dispensed in such a way that the substance is conveyed to the free end of the wick by the capillary action of the wick. An electrical heating element is regularly assigned to the free end of the wick, by means of which heat can be applied to the free end of the wick in order to be able to release the substance accumulating in the free end of the wick into the environment or evaporate it even faster. Such a structure is known from WO 98/58692 A1, for example. In order to be able to adjust the degree of evaporation and thus the evaporation capacity, said WO 98/58692 A1 also provides for the container and wick to be stored in the housing of the device in such a height-adjustable manner that the relative position of the wick to the heating device can be changed.

With such a design, the evaporation rate and thus the release rate of the substance to be released can be influenced over a longer period of time. However, there is the problem that the air flow generated and enriched with the substance to be dispensed, which escapes into the environment via an outlet opening in the housing wall, cannot flow out of the housing fast enough and is influenced, for example, by air turbulence arising within the housing, so that undesirable deposits and condensation of the substance to be dispensed on the inner walls of the housing occur to a certain extent. Furthermore, with such devices, especially if they are arranged close to a wall, there is a risk that the outflowing substance air flow will change into the desired unstable convection state just above the device. In a relatively low room, e.g. a device mounted on a wall near a socket, this often leads to an insufficiently effective distribution of the substance to be emitted in the room or to undesired deposits of the substance to be emitted on the wall.

Accordingly it is an object of the present invention to create a device and a method for the dispensing and diffusing, in particular for the evaporation of volatile substances, by means of which an effective outflow of the substance air flow can be ensured or a sufficient distribution of the substance to be delivered in the room can be achieved.

This object is solved with the features of the independent patent claims. Advantageous embodiments are subject of the sub-claims.

The invention relates to a device for dispensing and/or diffusing volatile, e.g. liquid or gel substances, in particular for dispensing and/or diffusing fragrances and/or active substances, with a housing surrounding an interior space of the housing. Furthermore, the device has at least one storage tank in which a substance to be released into the environment is contained. Furthermore, at least one electrical heating device is provided, which is accommodated in the interior of the housing and has an electrical heating element, preferably formed by a heating wire or a heating coil, by means of which heat can be generated for heating and evaporating the substance to be released.

Furthermore, at least one capillary element is provided, which is directly or indirectly coupled to the heating element in a heat-transferring manner, wherein the capillary element has at least one substance-receiving area which is in contact with the substance received in the storage tank, so that this substance can be conveyed from the substance-receiving area by means of capillary action to a substance-discharging area of the capillary element, where the substance is discharged.

At least one air inlet on the housing side is provided, by means of which ambient air enters from outside the housing and forms a pure flow in the housing interior. Preferably, an air generating device is provided, by means of which the ambient air can be drawn in from outside the housing via the at least one air inlet on the housing side in order to generate said pure flow.

The device further comprises at least one air duct which, starting from the at least one air inlet, is guided to the substance-discharging area of the capillary element, where the pure air flow is enriched with the substance and a substance air flow is generated, and which, starting from the substance-discharging area, is led to at least one air outlet on the housing side, via which the substance air flow can be discharged to the environment.

Furthermore, at least one control device is provided, by means of which at least the at least one electrical heating device can be controlled during operation of the device, preferably at intervals. Preferably, at least one control device is provided by means of which the at least one air generating device and the electrical heating device can be controlled during operation of the device, preferably at intervals.

With such a device, a targeted substance air flow can be generated, which is not only effectively and quickly enriched with substance, but also releasable into the environment at a high flow rate. This depends on the one hand, on the evaporator temperature set by means of the electrical heating device, but also on the flow velocity of the substance air flow generated. As the tests carried out by the inventor have shown, the forced flow generated for example by means of an air generating device, in conjunction with a high substance release rate caused by the vaporization, can cause a particularly effective and rapid filling of the room with the substance or a sufficiently good distribution of the substance in the room.

In this context, the method according to the invention will also be discussed in detail:

Particularly preferred and advantageous is a method control, in which the at least one electrical heating device, preferably the at least one air generating device and the at least one electrical heating device, is respectively are controlled by means of the control device in such an interval-like manner that during each interval a substance quantity is delivered by the device which is equal or smaller than the substance quantity temporarily stored in the capillary element. Alternatively or additionally it can be provided that the capillary element is enriched in the time between the individual intervals with a substance quantity which is equal or larger than the substance quantity delivered in the next interval.

Both procedures ensure that the capillary element is always moist or wet during the heating process, so that, for example, a heating coil as an electrical heating element never acts only on a dry capillary element, which can have a negative impact on the durability and life of both the capillary element and the electrical heating element. For this reason it is also advantageous, in connection with the solution according to the invention, to use only low heating temperatures between 100° C. and 280° C., preferably between 100° C. and 250° C.

Another positive factor in preventing overheating of the capillary element and/or the electrical heating element is that the pure flow generated by for example by means of the air generating device has a certain cooling function.

The inventor-side tests have further shown that the heating temperature and the speed of the air flow should preferably be adjusted so that the size of the substance particles released into the environment is 20 to 80 Nm, preferably 30 to 60 Nm.

A further particular advantage of the device according to the invention is that it can also be used to evaporate non-solution-based substances, such as essential oils, thus significantly reducing the amount of VOC in the air.

In accordance with another particularly preferred embodiment, it is intended that the electrical heating device forms a structural unit, in particular a pinheater structural unit, which comprises the electrical heating element and the capillary element. Here, the electric heating element is in direct contact with the capillary element, which considerably improves the heat transfer between the electric heating element and the capillary element. For example, at least a part of the electrical heating element can even be embedded in the capillary element.

The electric heating element, preferably formed by a heating coil with several turns or windings, is preferably coupled to a power supply device which supplies the electric heating element with energy and heats it, controlled by the control device. For example, the energy supply may be a battery or accumulator. Plug or cable solutions, for example for an energy supply via a socket, are of course also possible at any time.

According to a specific embodiment, the housing is intended to have or form a heating area in which the electrical heating device is accommodated, preferably approximately in the middle or center of the heating area. With this design, the capillary element forming part of the electric heating device is in direct or indirect contact with the substance in the storage tank. The capillary element, on the other hand, lies with its substance discharging area in the flow path of the pure air flow flowing over the air duct, so that the pure air flow can be enriched with the substance as it flows over the substance delivery area. A design in which the capillary element area of the capillary element, which has and/or forms the substance delivery area, protrudes upwards in an approximately tower-like manner when viewed in the vertical axis direction is particularly preferred here.

Preferably, the capillary element region is covered from above by a covering element, preferably by a covering element which, viewed in the vertical axis direction, forms an upper housing wall and/or closes the housing at the top. The covering element defines a gap between the substance delivery region and the covering element, with said gap being a component of the air duct. The cover element has at least one opening, preferably forming the housing-side air outlet. Preferably, the cover element has at least one air outlet in the region above the tower top of the tower-like capillary element region. It is especially preferred that the cover element and/or the gap between the cover element and the substance delivery region surround(s) the substance delivery region at least partially, preferably fully, viewed in the circumferential direction of the substance delivery region.

With such a concrete design, an outflow nozzle is created in the area around the capillary element, whereby it is ensured in a simple and effective manner that the pure air flow flowing into the nozzle area is quickly and effectively enriched with the substance to be dispensed, so that a substance air flow with a sufficiently high velocity then flows out of the device via the opening and there creates a desired large or high distribution rate of the substance in a room.

As already mentioned above, it is advantageous that at least the inner side of the cover element, building the gap and/or being adjacent to the capillary element, especially adjacent to the substance delivery region of the capillary element, has a nozzle shape tapering in the direction of the opening, preferably with a central opening as an air outlet on the housing side.

As further shown by the inventor's tests, it is particularly advantageous if the cover element, which has a nozzle shape, is surrounded by a circumferential collar on the outer side facing away from the capillary element. This collar further stabilizes the substance air flow emerging from the housing.

The capillary element can, in accordance with a particularly preferred design, act as a first capillary element in conjunction with a further, second capillary element, preferably a wick, which is arranged in the storage tank and conveys the substance to be dispensed from the storage tank by means of capillary action. The interaction preferably takes place in such a way that a substance-receiving area of the first capillary element is directly or indirectly connected to a free end of the second capillary element. This design has the advantage that, for example, commercially available containers with a wick as the second capillary element can be used, which then ultimately only need to be coupled to the first capillary element to transfer substance. For this coupling, it has proven to be particularly advantageous if the first capillary element and the second capillary element are in active connection with the interposition of a porous ceramic disc. This results in an advantageous stabilization of the arrangement as a whole and also ensures the transfer of substances, especially when liquids are the preferred substances.

The storage tank of this embodiment, as already explained above, is preferably formed by a container, preferably an exchangeable container, which is held in a holding device located inside the housing. For exchangeability, the holder is of course designed as a detachable holder.

To ensure a particularly effective air flow inside the housing, the container is accommodated in the interior of the housing in such a way that, viewed in the circumferential direction, the container is at least partially spaced from the housing wall by a gap. In this case, the gap is a component of the air duct through which the pure air flow flows to the substance-discharging area, whereby it is preferably provided that the pure air flow (preferably a pure air flow drawn in by the at least one air generating device is taken in via the at least one housing-side air inlet in a lower housing area, preferably in the bottom wall) flows upwards along the container to the substance-discharging area of the capillary element and from there flows as a substance-enriched substance air flow across the gap between the cover element and the capillary element area to the opening of the cover element forming the housing-side air outlet and further into the environment.

In connection with the latter exemplary design, it is also advantageous if the at least one air generating device and/or the control device and/or a power supply device are located in the area below the container.

The housing may also have a bottom wall, preferably a bottom wall which closes the housing at the bottom and/or which is removable for access to the housing interior and/or which comprises at least one air inlet on the housing side. This results in an overall compact design which is also not very critical from a safety point of view, as any intervention in the interior of the housing containing the air generating device is avoided.

For a particularly compact design, the control unit can also be part of a printed circuit board (PCB) accommodated in the housing.

For ease of replacement, the electrical heating device may also be interchangeably mounted in the housing.

Since the preferred design provides that the capillary element is in direct contact with the electric heating element, it is particularly advantageous if the capillary element is made of a porous ceramic and/or a porous plastic.

The air generating device itself can be designed in different ways. Particularly advantageous are designs in which the air generating device is formed by at least one fan, for example a fan wheel. But also an air generating device which is formed by at least one air pump can be used, for example in the form of a piezo-controlled air pump.

The invention is described in more detail below by means of a drawing:

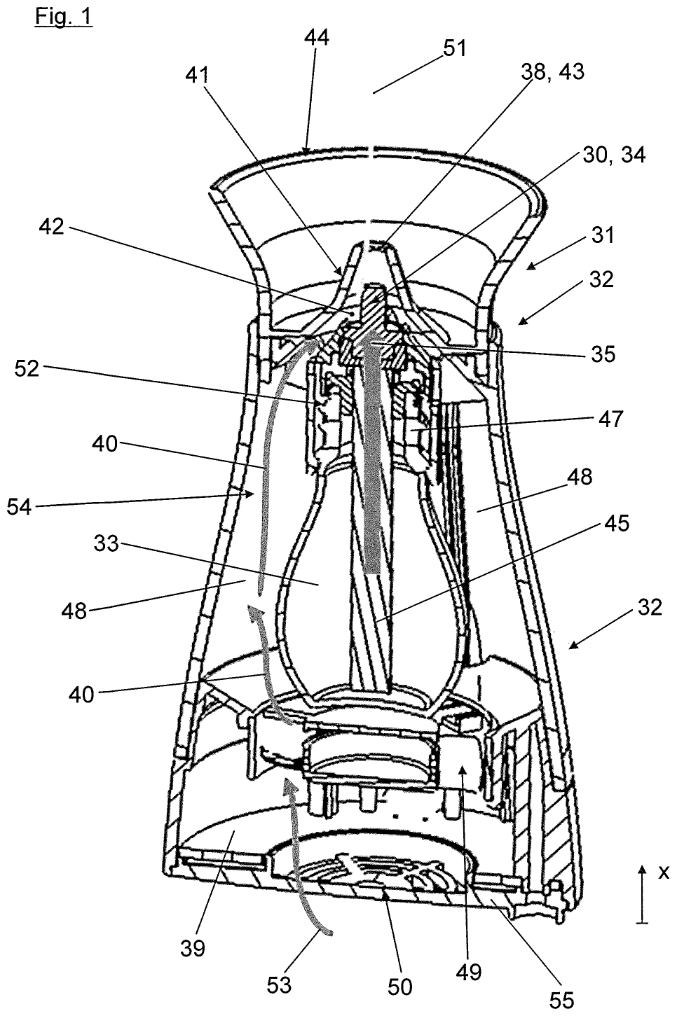

FIG. 1 a schematic, partially cut-open and perspective sketch of an exemplary first type of a device for dispensing and/or diffusing volatile substances according to the invention, and

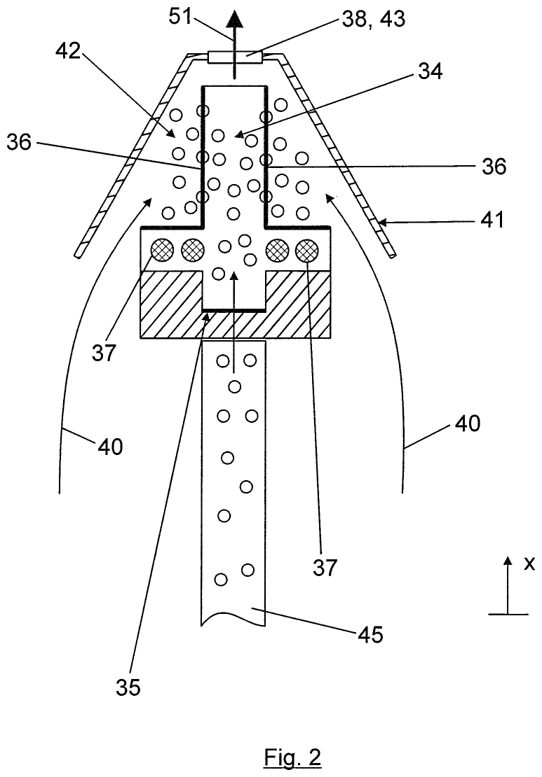

FIG. 2 a schematic sketch of the operation and structure of the electric heating device of the device of FIG. 1, which is essentially formed by a capillary element and a heating coil.

FIG. 1 shows, as an example and schematically, a partially cut-open, perspective representation of a device for dispensing volatile substances, for example for dispensing fragrances and/or active substances, in accordance with the invention, in which the housing 32 has a heating area 31 in which an electrical heating device 30 is accommodated, here exemplarily approximately in the middle.

A capillary element 34, which forms part of the electric heating device 30, has its substance receiving area 35 (see FIG. 2) in indirect contact with a substance accommodated in the storage tank 33, which is described in more detail below:

The capillary element 34 with its substance discharging area 36 is located in the flow path of the pure air flow 40 flowing over an air duct 54 (here only drawn very schematically and exemplarily), so that the pure air flow 40 can be enriched with the substance when flowing over the substance discharging area 36.

The upper capillary element area of the capillary element 34, which forms the substance discharging area 36, here exemplarily seen in vertical axis direction x, protrudes upwards, approximately like a tower, whereby this tower-like, upper capillary element area is laterally surrounded by a cover element 41 and covered from above, forming a gap 42 running around the circumference. Here, the cover element 41 forms an upper housing wall, viewed in vertical axis direction x, which closes the housing at the top.

The gap 42 is also part of the air duct 54, which is also described in more detail below.

The cover element 41 has a nozzle shape tapering towards an opening 43, whereby the opening 43 is designed as a central opening which simultaneously forms an air outlet 38 on the housing side.

The nozzle-shaped cover element 41 is also surrounded by a collar 44 (see FIG. 1) on the outer side facing away from the capillary element 34.

The capillary element 34 here also forms an example of a first capillary element which interacts with a further, second capillary element 45 (here formed by a wick) arranged in the storage tank 33, which conveys the substance to be dispensed from the storage tank 33 by means of capillary action. In this case, a substance receiving area 35 of the first capillary element 34, here indirectly with the interposition of a porous ceramic disc 46, is in operative connection with a free end of the second capillary element 45.

Here, the storage tank 33 is formed by an exchangeable container, which is preferably detachably mounted in a holding device 47 located inside the housing.

The container forming the storage tank 33 is accommodated in the interior of the housing 32 in such a way that the container, viewed in the circumferential direction, is at least partially, here by way of example fully spaced from a housing wall 56 by a gap 48.

The gap 48 is part of the air duct 54, through which the pure air flow 40 flows to the substance discharging area 36, and here only as an example in such a way that the ambient air drawn in by a fan 49 flows through a housing-side air inlet 50 formed in a bottom wall 55, seen in vertical axis direction x, into the housing interior and further flows, as the pure air flow 40, upwards along the container (see FIG. 2) to the substance discharging area 36 of the capillary element 34. From there it flows as a substance-enriched substance air flow 51 via the gap 42 between the cover element 41 and the capillary element area to the opening 43 of the cover element 41 forming the air outlet 38 on the housing side and further into the environment.

As is only exemplarily shown in FIG. 1, the fan 49 and the control device 39 as well as a power supply device 52 are arranged here in the area below the container 33.

The control device 39 can also be part of a printed circuit board (PCB) accommodated in the housing 32. Likewise, the electrical heating device 30 can be mounted interchangeably in the housing 56.

As already mentioned, the housing 32 has, as an example, a bottom wall 55, which can be removed, e.g. for access to the interior of the housing, and in which the air inlet 50 is located.

The capillary element 34 can be made of a porous ceramic and/or a porous plastic, for example.

The functioning of this exemplary inventive embodiment is as follows:

When the device is actuated, the control device 39 controls the fan 49 and the electric heating device 30 or the heating coil 37 at intervals, i.e. for a certain preset period of time. This causes the capillary element 34 to heat up and the substance temporarily stored in the capillary element 34 due to capillary action, which is diffused in via the substance receiving area 35, evaporates via the substance-discharging area 36 into the area of the gap 42, into which the pure air flow 40 drawn in via the air inlet 50 flows.

Depending on the type and winding of the heating coil, a certain temperature can be preset for the respective substance, preferably preset in such a way that the capillary element 34 does not dry out, i.e. it is always ensured that only a substance quantity is delivered which is smaller than the substance quantity temporarily stored in the capillary element 34.

Instead of a fan 49 as an air generating device, an air pump can also be used, for example a piezo-controlled air pump, which is not shown here, however.

With the exemplary design it is preferred that the temperature of the heating coil 37 is preferably in the range between 100° C. and 250° C. and/or that during operation of the device, preferably in intermittent operation of the device, no substance quantity is discharged which is greater than the substance quantity temporarily stored in capillary element 34. This means, conversely, that the capillary element 34 must be able to accumulate a quantity of substance in the time between the individual intervals which is equal or greater than the quantity of substance released in the next interval.

With the device according to the invention, substance particles are preferably emitted into the environment with a size of for example 20 to 80 Nm, preferably to 60 Nm.

### LIST OF REFERENCE SIGNS

30 electric heating device
31 heating area
32 housing
33 storage tank
34 capillary element
35 substance receiving area
36 substance discharging area
37 heating coil
38 air outlet
39 control device
40 pure air flow
41 cover element
42 gap
43 opening
44 collar
45 second capillary element
46 ceramic disc
47 holding device
48 gap
49 fan
50 air inlet
51 substance air flow
52 energy supply device
53 ambient air
54 air duct
55 bottom wall
56 housing wall

1. Device for dispensing and/or diffusing volatile substances, in particular for dispensing and/or diffusing fragrances and/or active substances, said device comprising:
housing that surrounds a housing interior,
at least one storage tank containing a substance to be released into the environment,
at least one electrical heating device which is accommodated in the housing interior and has an electrical heating element by means of which heat is generated for heating and evaporating the substance to be emitted,
at least one capillary element, which is coupled to the heating element in a indirect heat-transferring manner, wherein the capillary element has at least one substance-receiving area which is in contact with the substance received in the storage tank, so that the substance is conveyed from the substance-receiving area by means of capillary action to a substance-discharging area of the capillary element, at which the substance is discharged,
least one air inlet on the housing side, by means of which ambient air enters from outside the housing and forms a pure air flow in the housing interior,
at least one air duct which, starting from the at least one air inlet, is guided to the substance-discharging area of the capillary element, at which the pure air flow is enriched with the substance and a substance air flow enriched with substance is generated, and which, starting from the substance-discharging area, is guided to at least one air outlet on the housing side, via which the substance air flow is discharged to the environment, and
at least one control device by means of which the actuation of the at least one electrical heating device is controlled during operation of the device.

2. The device according to claim 1, further comprising an air flow generation device, by means of which ambient air is drawn in from outside the housing via the at least one air inlet and the pure air flow is generated.

3. The device according to claim 2, wherein the at least one control device also controls the actuation of the at least one air flow generation device during operation of the device.

4. The device according to claim 1, wherein the electrical heating device forms a structural unit, which comprises the electrical heating element and the capillary element in such way that the electrical heating element is indirect contact with the capillary element and/or that least a partial area of the electrical heating element is embedded in the capillary element.

5. The device according to claim 1, wherein the electric heating element is formed by a heating coil with several turns or windings, and is coupled to a power supply device, which supplies the electric heating element with power and heats it, controlled by means of the control device.

6. The device according to claim 1, wherein
the housing includes a heating area in which the electric heating device is accommodated,
wherein the capillary element forms part of the electric heating device and is, with its substance receiving area, in direct or indirect contact with the substance in the storage tank, and
wherein the capillary element is, with its substance discharging area, located in the flow path of the pure air flow flowing over the air duct, so that the pure air flow is enriched with the substance as it flows over the substance discharging area.

7. The device according to claim 6, wherein the capillary element region of the capillary element which includes the substance delivery region projects upwards in an approximately tower-like manner, as viewed in a vertical axis direction.

8. The device according to claim 1, wherein
the capillary element region is covered from above by a covering element which, viewed in a vertical axis direction, forms an upper housing wall and/or closes the housing at the top,
wherein the covering element defines a gap between the substance delivery region and the covering element, with said gap being a component of the air duct, and
wherein the cover element has at least one opening forming the housing-side air outlet.

9. The device according to claim 8, wherein the cover element has at least one air outlet in the region above the tower top of the tower-like capillary element region.

10. The device according to claim 8, wherein the cover element and/or the gap between the cover element and the substance delivery region surround(s) the substance delivery region at least partially, as viewed in a circumferential direction of the substance delivery region.

11. The device according to claim 8, wherein at least an inner side of the cover element, building the gap and/or being adjacent to the capillary element, especially adjacent to the substance delivery region of the capillary element, has a nozzle shape tapering in the direction of the opening with a central opening as an air outlet on the housing side.

12. The device according to claim 11, wherein the cover element providing the nozzle shape is surrounded by a circumferential collar on an outer side facing away from the capillary element.

13. The device according to claim 1, wherein the capillary element includes a first capillary element that cooperates with a further, second capillary element arranged in the storage tank, which conveys the substance to be dispensed from the storage tank by means of capillary action, so that a substance-receiving area of the first capillary element is in operative connection with a free end of the second capillary element.

14. The device according to claim 13, wherein the first capillary element and the second capillary element are in operative connection with the interposition of a porous ceramic disc.

15. The device according to claim 1, wherein the storage tank is formed by a container, which is detachably held in a holding device arranged in the interior of the housing.

16. The device according to claim 15,
wherein the container is accommodated in the interior of the housing in such a way that the container, viewed in a circumferential direction, is spaced apart from the housing wall defining a gap therebetween, and
in that the gap is a component of the air duct, via which the pure air flow flows to the substance discharging region, so that the pure air flow sucked in by the at least one air flow generating device via the at least one housing-side air inlet in a lower housing area flows upwards along the container to the substance discharging area of the capillary element and from there flows as a substance-enriched substance air flow across the gap between the cover element and the capillary element area to the opening of the cover element forming the housing-side air outlet and further into the environment.

17. The device according to claim 16, wherein the at least one air flow generation device and/or the control device and/or a power supply device is or are arranged in the area below the container, viewed in a vertical axis direction.

18. The device according to claim 1, wherein the housing has a bottom wall which closes the housing downwards and/or which is removable for access to the interior of the housing and/or which comprises at least one housing side air inlet.

19. The device according to claim 1, wherein the control device is part of a printed circuit board (PCB) accommodated in the housing.

20. The device according to claim 1, wherein the electric heating device is mounted in the housing in an exchangeable manner.

21. The device according to claim 1, wherein the capillary element is made from material selected from the group consisting of a porous ceramic and a porous plastic.

22. The device according to claim 1, wherein the airflow generation device is selected from the group consisting of a fan or an air pump.

23. The device according to claim 1, wherein
the at least one electrical heating device and the at least one air flow generation device are controlled in intervals by means of the control device so that during each interval a quantity of substance is delivered by the device which is equal or smaller than the quantity of substance temporarily stored in the capillary element,
wherein the capillary element accumulates in the time between the individual intervals with a quantity of substance which is equal or larger than the quantity of substance delivered in the next interval.

24. The device according to claim 23, wherein a maximum temperature of the heating element of the electrical heating device is between 100° C. and 280° C.

25. The device according to claim 23, wherein the size of the substance particles released and diffused into the environment is 20 to 80 nm.
